(19) United States
(12) Patent Application Publication
(10) Pub. No.: US 2009/0200240 A1
(43) Pub. Date: Aug. 13, 2009

(54) METHOD OF EXTRACTING DOMOIC ACID BY SELECTIVE ADSORPTION USING A HALOGENATED CARBOXYLIC ACID DERIVATIVE

(76) Inventors: Sergey Anatoliyovich Piletsky, Cranfield (GB); Olena Volodimirivna Piletska, Cranfield (GB); Fernando Navarro Villoslada, Madrid (ES)

Correspondence Address: STITES & HARBISON PLLC, 1199 NORTH FAIRFAX STREET, SUITE 900, ALEXANDRIA, VA, 22314, US

(21) Appl. No.: 12/162,193

(22) PCT Filed: Jan. 29, 2007
(86) PCT No.: PCT/GB07/00289

§ 371 Date: Jan. 14, 2009

(30) Foreign Application Priority Data
Jan. 28, 2006 (GB) 0601760.2

(51) Int. Cl. C02F 1/28

(52) U.S. Cl. 210/692; 210/691

(57) ABSTRACT

Domoic acid is selectively bound by an adsorbent which comprises units derived from one or more halogenated carboxylic acids (preferably having an α-trihalomethyl group) or derivatives thereof. The adsorbent may be a polymer, or a solid support onto which the carboxylic acid, derivative or polymer has been grafted. The preferenced acid is 2-trifluoromethyl acrylic acid. Domoic acid can be selectively bound from a mixture (e.g. seafood extract, seawater or urine) in the presence of potential interferents such as aminoacids, and recovered using an eluant comprising an organic acid.

### TECHNICAL FIELD

Broadly, the present invention relates to the preparation of materials capable of binding domoic acid, and their use, e.g. in analytical chemistry, or environmental, clinical or food analysis. Preferred adsorbents have a high affinity to domoic acid and can be used for purification of this compound, extraction and enrichment from a variety of samples.

Domoic acid is a very dangerous neurotoxin which is produced by diatom algae Pseudo-nitzschia and accumulated by shellfish and other marine species during algal blooms. Ingestion of domoic acid contaminated shellfish may lead to amnesic shellfish poisoning, which has caused death in both animals and humans in severe cases. For this reason, monitoring of domoic acid levels in both environmental samples in or near fisheries and in shellfish for human consumption is an important food safety measure. The European Commission Directive 2002/226/EC introduced a maximum permitted level of 20 mg domoic acid equivalents/kg shellfish intended for human consumption. Similar maximum permitted levels have been adopted by most other food safety authorities. The detection of domoic acid requires a material which is able to adsorb the toxin specifically and strongly in order to pre-concentrate or purify it from interfering compounds, salts and other substances found in sea water, food samples and complex biological fluids. A preferred embodiment of the invention may provide an affinity matrix that could be used for the extraction, purification and pre-concentration of domoic acid in the quality which would be acceptable for any further detection and quantifications methods such as chromatography, including high-pressure liquid chromatography (HPLC) and HPLC in combination with mass-spectrometry (MS), gas chromatography and enzyme-linked immunoadsorbent assay (ELISA).

### BACKGROUND ART

REFERENCES

| Patent | Country | Issued | Title |
|---|---|---|---|
| U.S. Pat. | USA | Apr. 26, 1994 | Detection of contaminants |
| No. 5,306,466 | | | in food |

### OTHER REFERENCES

1. K. J. James, M. Gillman, M. F. Amandi, A. López-Rivera, P. F Puente, M. Lehane, S. Mitrovic, A. Furey. Toxicon, 46, 2005, 852-858.
2. E. R. Tor, B. Puschner, W. E. Whitehead. J. Agric. Food Chem., 51, 2003, 1791-1796.
3. http://www.jtbaker.com/techlib/docunients/8066.html
4. (Kotoulas C., Kiparissides C. (2006). A generalized population balance model for the prediction of particle size distribution in suspension polymerization reactors. Chemical Engineering Science, 61, 332-346
5. Li K., Stöver H. D. H. (1993). Synthesis of monodisperse poly(divinylbenzene) microspheres. J. Polym. Sci., A, Polym. Chem., 31, 3257-3263
6. Yasuda M., Seki H., Yokoyama H., Ogino H., Ishimi K., Ishikawa H. (2001). Simulation of a particle formation stage in the dispersion polymerization of styrene Macromolecules, 34, 3261-3270
7. Larpent C., Bernard E., Richard J., Vaslin S. (1997). Polymerization in microemulsions with polymerizable cosurfactants: a route to highly functionalized nanoparticles. Macromolecules, 30, 354-362
8. Koh K., Ohno K., Tsujii Y., Fukuda T. (2004). Synthesis of well-defined polymers with protected silanol groups by atom transfer radical polymerization and their use for the fabrication of polymeric nanoparticles. European Polymer Journal, 40, 2665-2670
9. Delfour M., Bennevault-Celton V., Nguyen H. A., Macedo A., Cheradarne H. (2004). Cationic polymerization of dienes VII. New electron donors in the polymerization of 1,3-pentadiene initiated by aluminum trichloride in non-polar solvent. European Polymer Journal, 40, 1387-1398).
10. Tsafack M. J., Levalois-Grützmacher J. (2006). Flame retardancy of cotton textiles by plasma-induced graft-polymerization (PIGP). Surface and Coatings Technology, 201, 2599-2610
11. Hu M.-X., Yang Q., Xu Zh.-K. (2006). Enhancing the hydrophilicity of polypropylene microporous membranes by the grafting of 2-hydroxyethyl methacrylate via a synergistic effect of photoinitiators. Journal of Membrane Science, 285, 196-205
12. Piletsky S. A., Matuschewski H., Schedler U., Wilpert A., Piletska E. V., Thiele T. A., Ulbricht M. (2000). Surface functionalization of porous polypropylene membranes with molecularly imprinted polymers by photo-graft copolymerization in water. Macromolecules, 33, 3092-3098.

Several materials have been used in the past for solid phase extraction (SPE) of domoic acid including: strong anion exchange (SAX) cartridges [1]; Oasis HLB extraction cartridge based on poly(divinylbenzene-co-N-vinylpyrrolidone), which exhibits both hydrophilic and lipophilic retention characteristics [2]; and strong cation exchange (CAX) cartridges such as BAKERBOND Spe™, based on aromatic sulfonic acid [3]. They all have some disadvantages related to their operational mode. SAX cartridges and Oasis HLB adsorbents have not been shown to possess sufficiently high affinity for binding low (<1 ng/ml) and high (>500 μg/ml) concentrations of domoic acid. An additional problem is the inability of the commercial adsorbents to discriminate between domoic acid and amino acids such as aspartic and glutamic acids. The immunoadsorbents described in U.S. Pat. No. 5,306,466 in many instances possess the required affinity and selectivity but suffer from poor stability and high cost. Strong cation exchange (CAX) cartridges containing aromatic sulfonic acid have other disadvantages arising from the complicated recovery of domoic acid adsorbed by these materials. Thus to remove domoic acid from these adsorbents it would be required to use strong mineral acid such as sulfuric or hydrochloric acids. This is poorly compatible with many applications such as MS detection or ELISA.

### DISCLOSURE OF THE INVENTION

The invention provides a method of extracting domoic acid from a mixture containing it comprising: (a) preparing an adsorbent that selectively binds domoic acid; and (b) contacting said adsorbent with a solution of, or derived from, said mixture so that domoic acid is adsorbed; wherein said adsorbent comprises units derived from one or more halogenated carboxylic acids or derivatives thereof. The term “derivatives” encompasses salts, esters, thioesters, amides and nitriles. The carboxylic acid moiety is preferably halogenated at least at a carbon atom D to the carbonyl atom of the carboxylic acid group (or the corresponding position in derivatives). Preferably there is an α-trihalomethyl group. More preferably it is α-trichloromethyl or (most preferably) α-trifluoromethyl.

Preferred carboxylic acids are of formula (A) or (B), and preferred derivatives are derivatives thereof:

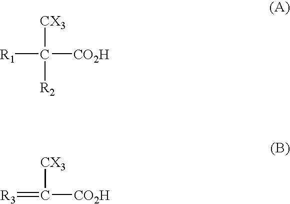

where X is a halogen atom,
R₁ is selected from hydrogen, optionally-substituted alkyl or cycloalkyl, optionally-substituted aryl or heteroaryl, optionally-substituted amine, —OR or —SR where R is alkyl, cycloalkyl, aryl or heteroaryl and is optionally substituted;
R₂ is selected from optionally-substituted alkyl or cycloalkyl, optionally-substituted aryl or heteroaryl, optionally-substituted amine, —OR or —SR where R is as defined above, or is a group containing a double or triple carbon-carbon bond; and
R₃ is selected from optionally-substituted alkylene or cycloalkylene, oxygen and sulphur.

Alkyl and alkylene groups generally have 1-6 C atoms. Aryl and heteroaryl generally have up to 12 C atoms. “Optionally substituted” refers to the possibility that one or more H atoms are replaced by a substituent suitably selected from halogen, OR, SR, NR₂, —CO—R, aryl or heteroaryl where R is as defined above.

If the carboxylic acid does not contain a carbon-carbon multiple bond, use will generally be made of a derivative which does, e.g. an ester of vinyl or allyl alcohol.

In some contexts, use may be made of a carboxylic acid or derivative that is a variant of formula (A) where—CX₃ is replaced by H. The compound may not then be halogenated. For example it may be diethylaminoethyl methacrylate (DEAEM).

A method embodying the invention may be used inter alia in analytical chemistry, and in environmental, clinical and food analysis.

Preferred materials capable of binding domoic acid are constructed from trifluoromethyl carboxylic acids and their derivatives, such as 2-trifluoromethyl acrylic acid. Adsorbents synthesised from these derivatives can have a high affinity for domoic acid and can be used for the extraction, enrichment and purification of this compound. The substantial advantage of proposed materials includes relatively easy recovery of domoic acid from the adsorbents by elution with acidified organic solvent, such as, for example, a solution of formic acid or acetic acid in acetonitrile or methanol. Other alternative organic acids and organic solvents will readily occur to those skilled in the art. The proposed adsorbents allow for the selective extraction of domoic acid from mixtures containing structural analogues, including but not limited to other amino acids such as glutamic and aspartic acids, glutamine, proline, gamma-aminobutyric acid (GABA) and kainic acid as well as other matrix components.

### MAIN EMBODIMENTS INCLUDE

(1). Synthesis of the adsorbents from polymerisable derivatives of halogenated, and more specifically fluorinated, carboxylic acids by polymerisation; optionally followed by:
(2). grafting of the resulting polymers containing halogenated, and (preferably fluorinated) carboxylic acids to the surface of beads, membranes or other supports.

The synthesised adsorbents may then be used for the extraction, enrichment, or purification of domoic acid or its detection.

### MODES FOR CARRYING OUT THE INVENTION

Adsorbents can be produced from polymerisable carboxylic acids or their derivatives. Carboxylic acid derivatives include, but are not limited to, inorganic or organic acid salts, esters, thioesters, amides, and nitrites. Normally a carboxylic acid (or derivative thereof) monomer, containing a polymerizable double bond is mixed together with cross-linker and radical initiator in an appropriate organic solvent or in water. Polymerisation can be initiated by heating, or preferably by UV irradiation and normally takes minutes or hours depending on the reactivity of the species. Several different forms of polymerisation may be employed, including radical polymerisation, living polymerisation, ionic polymerisation, suspension or emulsion polymerisation [see for example 4-9]. The preferred kind of polymerisation is a radical polymerisation. The monomers which can be used for adsorbent preparation include derivatives of carboxylic acids: vinyl monomers, allyl monomers, acetylenes, acrylates, methacrylates. The carboxylic acids are halogenated. More preferably the carboxylic acids are halogenated on a carbon atom beta to the carbonyl of the carboxylic acid group. Even more preferably the carboxylic acids carry an alpha tri-halomethyl group, and still more preferably carry an alpha tri-fluoromethyl group. Most preferred is 2-trifluoromethyl acrylic acid (TFMAA).

Typical examples of cross-linkers suitable for synthesis include, but are not limited to, ethylene glycol dimethacrylate, methylene bisacrylamide and N,N′-bisacryloylpiperazine. Those skilled in the art could select monomers and cross-linkers suitable for a particular system.

Adsorbents can also be prepared by grafting of carboxylic acids or their derivatives, or polymers thereof, preferably fluorinated, onto the surface of supports. Preferred supports include prepared articles such as pre-formed beads, membranes, capillaries or fibres. Grafting could be achieved in a variety of ways known to practitioners in the art [see for example 10-12]. Thus the surface of corresponding inorganic or polymeric materials can be activated with functional groups capable of covalent attachment of corresponding carboxylic acids, derivatives of carboxylic acids, or polymers thereof. Examples of chemical reaction used for this include the formation of Schiff's bases, disulfide bonds, S-metal bond, or formation of esters. Another type of grafting could include radical grafting with radical initiator immobilised on the surface of beads or membranes. Plasma grafting also can be used for production of affinity adsorbents.

The synthesised adsorbents may be used for the purification of domoic acid, or its extraction or enrichment from a variety of samples, or its detection and/or quantification. Application of the synthesised absorbents for detection or quantification of domoic acid may be performed in a variety of ways, including but not limited to assay of the eluate from the absorbents by analytical techniques well known to those skilled in the art, or the absorbent may be used as the detection matrix in a chemical sensor device. A typical example of this application could be solid phase extraction of domoic acid from seawater, biological or food samples. These adsorbents can be used in combination with quantification methods such as chromatography, including high-pressure liquid chromatography (HPLC) and HPLC in combination with mass-spectrometry (MS), gas chromatography and enzyme-linked immunoadsorbent assay (ELISA) or sensors.

The present invention will now be further described particularly with reference to the following non-limiting examples.

### Example 1

### Synthesis of Cross-Linked 2-Trifluoromethyl Acrylic Acid (TFMAA)

The polymer was synthesised by mixing 100 mg of TFMAA, 2 ml of DMF, 900 mg of cross-linker, ethylene glycol dimethacrylate (EGDMA) and 10 mg of azobisisobutyronitrile (AIBN), as an initiator. The polymer mixture was thermo-polymerised in an oil bath at 80° C. for 12 h. After synthesis, polymer was ground and wet-sieved with methanol to obtain particles of 63-106 μm. The polymer (100 mg) was packed into 1 ml SPE cartridges and used for the adsorption of domoic acid. In order to remove the unreacted monomers, polymer cartridges were washed with 5 ml of 50% methanol containing 100 mM NaOH followed by 10 ml deionised water. A similar protocol was also used for regeneration of the cartridges. 0.1 M HCl was used for cartridge pre-conditioning before the experiment.

### Example 2

### Synthesis of Cross-Linked Functional Monomers

Polymers were prepared by mixing functional monomer (diethyl amino ethyl methacrylate (DEAEM), methacrylic acid (MAA) or TFMAA) (1 g), cross-linker (EGDMA) (4 g) and initiator (50 mg) in DMF (5 g). The polymer mixture was degassed for 2 min with nitrogen. The polymers were prepared using thermal polymerisation (80° C.) for 12 h. The polymers were ground manually and wet-sieved with methanol. The fraction between 45 and 65 μm was collected. Polymer particles were dried in the oven at 60° C. for 30 min. 1-ml SPE cartridges were packed with 50 mg of polymer. The binding properties of the polymers were tested at pH 4.6 (50 mM Na-phosphate buffer). The cartridges were conditioned with 10 ml of buffer before the experiment. 500 μl of domoic acid solution with concentration 65 μg/ml was passed through each cartridge, the filtrate collected and analysed using HPLC. The results show that the binding of domoic acid at pH 4.6 was 100% for diethyl amino ethyl methacrylate-based polymer (DEAEM), 80% for methacrylic acid based polymer (MAA) and 100% for 2-trifluoromethyl acrylic acid-based polymer (TFMAA). The binding of domoic acid from seawater (˜3.5% NaCl) was essentially the same for TFMAA but substantially reduced in the case of DEAEM and MAA based polymers (<40%).

### Example 3

### Application of TFMAA-Based Adsorbent in the Selective Extraction of Domoic Acid from Seawater

Artificial seawater was prepared as a 3.5%-solution of sea salt (Sigma, S-9883). The artificial seawater samples were then spiked with domoic acid, or structural analogues of domoic acid such as glutamic and aspartic acids. A set of experiments was conducted in order to evaluate their recovery from the TFMAA-based polymer. After conditioning of the cartridges with 1 ml 0.1 M HCl, artificial seawater, spiked with different concentrations of domoic acid (100-500 ng/mL, 0.3-1.6 μM), glutamic acid (29-147 μg/ml; 0.25-1 mM) and aspartic acid (26-133 μg/ml; 0.25-1 mM) was loaded onto the cartridges. The quantification of all three analytes—domoic acid, glutamic and aspartic acid, in the eluate was performed simultaneously using HPLC-MS. All samples demonstrated quantitative recovery of domoic acid and only negligible recovery (<1%) of glutamic and aspartic acids (Table 1).

TABLE 1
The recovery of domoic acid from an artificial seawater sample
spiked with domoic, glutamic and aspartic acids; 80% acetonitrile
containing 0.1 M formic acid was used as eluent.

| [Domoic | | [Glutamic | | [Aspartic | |
|---|---|---|---|---|---|
| acid] | Rec., % | acid] | Rec., % | acid] | Rec., % |
| 250 | 96 ± 6 | 147,000 | 0.3 ± 0.1 | 133,000 | 0.3 ± 0.1 |
| 100 | 92 ± 9 | 58,800 | 0.7 ± 0.2 | 53,200 | 0.6 ± 0.2 |
| 50 | 95 ± 4 | 29,400 | 1 ± 0.4 | 26,600 | 0.9 ± 0.3 |

The concentrations are given in ng/ml.

### Example 4

### Application of TFMAA-Based Adsorbent in Selective Extraction of Domoic Acid from Urine

Similarly, experiments with the extraction of urine samples spiked with different concentrations of domoic, glutamic and aspartic acids were carried out. It was found that a sufficient level of recovery for domoic acid was also achieved with urine samples (Table 2), the same time the recovery of glutamic and aspartic acid was low (<1-2%).

TABLE 2
Recovery of domoic acid from urine samples spiked
with domoic and glutamic acids; 80% acetonitrile
containing 0.1 M formic acid was used as eluant.

| [Domoic acid] | Recovery, % | [Glutamic acid] | Recovery, % |
|---|---|---|---|
| 250 | 90 ± 5 | 147,000 | 0.2 ± 0.1 |
| 100 | 82 ± 5 | 58,800 | 1 ± 0.1 |
| 50 | 89 ± 4 | 29,400 | 2 ± 0.2 |

The concentrations are given in ng/ml.

### Example 5

### Application of TFMAA-Based Adsorbent in Selective Extraction of Domoic Acid from Shellfish

Shellfish flesh (50 g) is homogenised in a high speed blender and samples of the homogenate (5 g) are mixed together with a 50% methanol/water extraction solution (20 ml) in a 50 ml centrifuge tube with a vortex mixer and then centrifuged at 3,000×g for 10 minutes at room temperature. The supernatant is removed and passed through a 1 ml SPE cartridge as above (pre-conditioned with 10 ml of buffer before the experiment). The domoic acid is eluted with 80% acetonitrile containing 0.1 M formic acid and the eluate is collected.

1) A method of extracting domoic acid from a mixture containing it comprising:
(a) preparing an adsorbent that selectively binds domoic acid; and
(b) contacting said adsorbent with a solution of, or derived from, said mixture so that domoic acid is adsorbed; wherein said adsorbent comprises units derived from one or more halogenated carboxylic acids or derivatives thereof.

2) A method according claim 1 wherein said adsorbent comprises units derived from one or more carboxylic acid derivatives selected from salts, esters, thioesters, amides and nitriles.

3) A method according to claim 1 wherein said carboxylic acid or derivative is halogenated at a carbon atom P to the carbonyl of the carboxylic acid group.

4) A method according to claim 3 wherein the carboxylic acid or derivative has an α-trihalomethyl group.

5) A method according to claim 1 wherein the carboxylic acid or derivative is fluorinated.

6) A method according to claim 5 wherein the carboxylic acid or derivative has an α-trifluoromethyl group.

7) A method according to claim 1 wherein said carboxylic acid is of formula A or B: 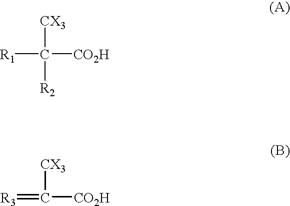
where X is a halogen atom,
R₁ is selected from hydrogen, optionally-substituted alkyl or cycloalkyl, optionally-substituted aryl or heteroaryl, optionally-substituted amine, —OR or —SR where R is alkyl, cycloalkyl, aryl or heteroaryl and is optionally substituted;
R₂ is selected from optionally-substituted alkyl or cycloalkyl, optionally-substituted aryl or heteroaryl, optionally-substituted amine, —OR or —SR where R is as defined above, or is a group containing a double or triple carbon-carbon bond; and
R₃ is selected from optionally-substituted alkylene or cycloalkylene, oxygen and sulphur;
said adsorbent having units derived from said carboxylic acid or a derivative thereof.

8. A method according to claim 7 wherein X is F or Cl.

9. A method according to claim 8 wherein said carboxylic acid is 2-trifluoromethyl acrylic acid.

10. A method according to claim 1 wherein said adsorbent comprises a polymer prepared by polymerising a monomer which is a carboxylic acid or derivative thereof having a carbon-carbon multiple bond which is employed in the polymerisation.

11. A method according to claim 10 wherein the polymerisation also involves a cross-linker.

12. A method according to claim 11 wherein the cross-linker is selected from the group consisting of ethylene glycol dimethacrylate, methylene bisacrylamide and N,N′-bisacryloyl piperazine.

13. A method according to claim 10 wherein said polymerising step employs free radical polymerisation.

14. A method according to claim 10 wherein said polymerising step-employs a process selected from living polymerisation, ionic polymerisation, suspension polymerization and emulsion polymerisation.

15. A method according to claim 1 wherein said adsorbent is prepared by grafting a carboxylic acid, a derivative thereof, or a polymer of a carboxylic acid or derivative thereof, onto a solid support.

16. A method according to claim 15 wherein said solid support is in the form of beads, fibres, capillaries or a membrane.

17. A method according to claim 1 including a subsequent step (c) of eluting domoic acid from the adsorbent.

18. A method according to claim 17 wherein said step (c) employs an eluant comprising an acid.

19. A method according to claim 18 wherein the eluant comprises an organic acid.

20. A method according to claim 17, including a subsequent step (d) of quantifying the eluted domoic acid.

21. A method according to claim 1 wherein said mixture comprises a biological or food sample.

22. A method according to claim 1 wherein said mixture comprises seawater, a homogenate or extract of fish or shellfish, or urine.

23. A method according to claim 1 wherein said mixture also contains one or more of glutamic acid, aspartic acid, glutamine, proline, γ-aminobutyric acid and kainic acid.
